(19) United States
(12) Patent Application Publication
(10) Pub. No.: US 2013/0158393 A1
(43) Pub. Date: Jun. 20, 2013

(54) Concentric Drive Scanning Probe

(71) Applicants: Michael Papac, North Tustin, CA (US); John Christopher Huculak, Mission Viejo, CA (US); Michael J. Yadlowsky, Sunnyvale, CA (US)

(72) Inventors: Michael Papac, North Tustin, CA (US); John Christopher Huculak, Mission Viejo, CA (US); Michael J. Yadlowsky, Sunnyvale, CA (US)

(21) Appl. No.: 13/692,307

(22) Filed: Dec. 3, 2012

Related U.S. Application Data
(60) Provisional application No. 61/577,379, filed on Dec. 19, 2011.

(51) Int. Cl. A61F 9/008; A61B 5/00

(52) U.S. Cl. CPC A61F 9/008; A61B 5/0066; A61B 5/0084
USPC 600/427; 606/17; 600/425

(57) ABSTRACT

An endoprobe for microsurgical procedures is provided, including a hand-piece having a concentric drive having a proximal coupling and a distal coupling and a cannula assembly coupled to the hand-piece. The cannula assembly may include an outer tube having a longitudinal axis and an inner tube positioned within the outer tube, the distal coupling providing a first rotation to the outer tube about the longitudinal axis and the proximal coupling providing a second rotation to the inner tube within the outer tube. According to embodiments disclosed herein a method for scanning a light beam using a cannula assembly as described above is also provided.

### PRIORITY CLAIM

This application claims the benefit of priority of U.S. Provisional Patent Application Ser. No. 61/577,379 titled “Concentric Drive Scanning Probe”, filed on Dec. 19, 2011, whose inventors are Michael D. Papac, John C. Huculak, and Michael J. Yadlowsky, which is hereby incorporated by reference in its entirety as though fully and completely set forth herein.

### BACKGROUND

1.—Field of the Invention

Embodiments described herein relate to the field of microsurgical probes. More particularly, embodiments described herein are related to the field of endoscopic Optical Coherence Tomography (OCT) and to the field of ophthalmic microsurgical techniques.

2.—Description of Related Art

The field of microsurgical procedures is evolving rapidly. Typically, these procedures involve the use of probes that are capable of reaching the tissue that is being treated or diagnosed. Such procedures make use of endoscopic surgical instruments having a probe coupled to a controller device in a remote console. Current state of the art probes are quite complex in operation, often times requiring moving parts that use complex mechanical systems. In many cases, an electrical motor is included in the design of the probe. Many prior art devices have a cost that makes them difficult to discard after one or only a few surgical procedures. Furthermore, the complexity of prior art devices leads generally to probes having cross sections of several millimeters. These probes are of little practical use for ophthalmic microsurgical techniques. In ophthalmic surgery, dimensions of one (1) mm (millimeters) or less may be used, to access areas of interest without damaging unrelated tissue.

Scanning mechanisms that allow time-dependent direction of light for diagnostic or therapeutic purposes have been used in endoscopic surgical instruments. These instruments typically use probes that provide imaging, treatment, or both, over an extended area of tissue without requiring motion of the endoscope relative to its surroundings. However, efforts to develop scanning endoprobes compatible with ophthalmic surgery have been slowed by the difficulty of providing a complex drive mechanism in a compact form factor and at a low cost. This is particularly true for forward-directed scanning probes that may require counter rotating shafts with fixed or controlled relative speeds.

### SUMMARY

According to embodiments disclosed herein an endoprobe for microsurgical procedures includes a hand-piece having a concentric drive having a proximal coupling and a distal coupling and a cannula assembly coupled to the hand-piece. The cannula assembly may include an outer tube having a longitudinal axis and an inner tube positioned within the outer tube, the distal coupling providing a first rotation to the outer tube about the longitudinal axis and the proximal coupling providing a second rotation to the inner tube within the outer tube.

According to embodiments disclosed herein a method for scanning a light beam using a cannula assembly may include providing a light beam through an axis of the cannula assembly, using a concentric drive in a hand-piece proximal to the cannula to provide a rotation to an inner tube and a rotation to an outer tube in the cannula (wherein each of the outer tube and inner tube may be hollow and have an optical element in its distal end), and controlling separately the rotation of the outer tube and the rotation of the inner tube using the concentric drive.

These and other embodiments of the present invention will be described in further detail below with reference to the following drawings.

### BRIEF DESCRIPTION OF THE DRAWINGS

FIG. 1 shows a partial cross-sectional view of a microsurgical endoprobe including a hand-piece having a concentric drive and a cannula assembly, according to some embodiments.

FIG. 2A shows a partial view of a distal motor and a cannula assembly, according to some embodiments.

FIG. 2B shows a partial view of a concentric drive, according to some embodiments.

FIG. 3 shows a partial cross-sectional view of a microsurgical endoprobe including a hand-piece having a concentric drive and a cannula assembly, according to some embodiments.

FIG. 4 shows a partial stylized view of a microsurgical endoprobe including a hand-piece having a concentric drive and a cannula assembly, according to some embodiments.

FIG. 5A shows a partial stylized view of a concentric drive and a cannula assembly, according to some embodiments.

FIG. 5B shows a partial view of a cross section of a slider coupling mechanism, according to some embodiments.

FIG. 6 shows a flow chart of a method for scanning a light beam using a cannula assembly, according to some embodiments.

In the figures, elements having the same reference number have the same or similar functions.

### DETAILED DESCRIPTION

Microsurgical procedures using endoscopic instruments according to the present disclosure may include a probe having a simple and cost-effective drive coupling mechanism. The probe may be a hand-held probe for direct manipulation by specialized personnel. In some embodiments, the probe may be designed to be controlled by a robotic arm or a computer-controlled device. Probes have a proximal end close to the operation controller (be it a specialist or a device) and a distal end, close to or in contact with the tissue. Probes according to embodiments disclosed herein may have small dimensions, be easy to manipulate from a proximal end, and minimally invasive to the surrounding tissue. In the distal end, the probe ends with a tip, from where the probe performs certain actions on a target tissue located in the vicinity of the tip. For example, the probe may deliver light from its tip, and receive light reflected or scattered from the tissue, coupled through the tip. The tip of the probe may include movable elements that enable the tip to perform its action.

Embodiments disclosed herein include a simple and compact drive mechanism for providing separate control of scanner elements in scanning endoprobes. Some embodiments include rotating scanning elements. Such endoprobes may include applications such as optical coherence tomography (OCT), single or multi-spot therapeutic laser delivery, and illumination functionality. Embodiments of endoprobes using rotating scanning elements as disclosed herein are able to provide one-dimensional (1-D) line scan capability. According to some embodiments, independent control of two scanning elements enables two-dimensional (2-D) lateral scanning of the beam on tissue. Further, by rotating a line scan in an OCT system some embodiments obtain a three-dimensional (3-D) scan (2-D beam scan plus 1-D depth scan), or selective pattern scanning. Such endoprobes may be used in conjunction with system software to enable control of the scan pattern through a graphical user interface, a footswitch, a voice command, or a handheld control. Simple and compact drive is accomplished by attaching directly to, or integrating cannula tubes into the output shafts of electric drive motors, reciprocating pneumatic motors, or fan propelled motors.

Embodiments consistent with the present disclosure may be as disclosed in detail in U.S. Provisional Patent Application No. 61/466,364 entitled “Pneumatically Driven Ophthalmic Scanning Endoprobe” by Michael J. Papac, Michael Yadlowsky, and John Huculak, filed on Mar. 22, 2011 which is hereby incorporated by reference in its entirety as though fully and completely set forth herein. Also, embodiments of counter-rotating mechanisms for cannula assemblies may be as disclosed in detail in U.S. Provisional Patent Application No. 61/434,942 entitled “Counter-rotating Ophthalmic Scanner Drive Mechanism,” by Michael Yadlowsky, Michael J. Papac, and John Huculak, filed on Jan. 21, 2011 which is hereby incorporated by reference in its entirety as though fully and completely set forth herein. Some embodiments consistent with the present disclosure may use drive mechanisms as disclosed herein in reciprocating configurations according to embodiments described in detail in U.S. Provisional Patent Application Ser. No. 61/577,371 entitled “Reciprocating Drive Optical Scanner for Surgical Endoprobes” by Michael Yadlowsky, Michael J. Papac, and John C. Huculak filed December 19, 2011 which is hereby incorporated by reference in its entirety as though fully and completely set forth herein.

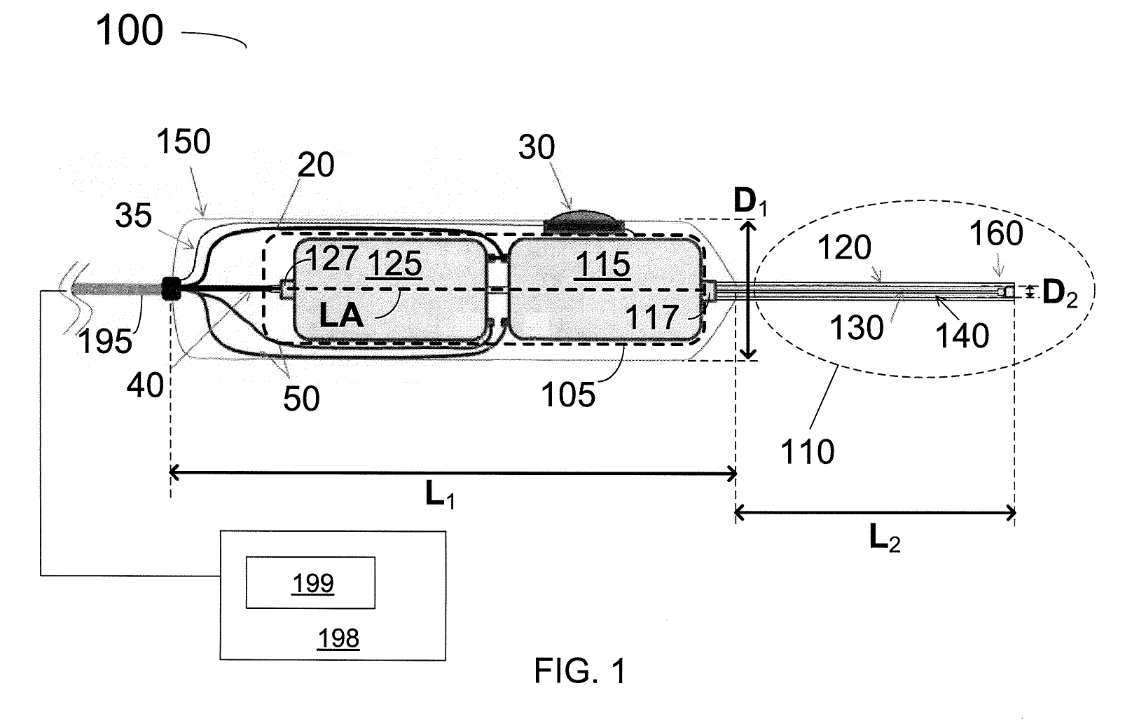

FIG. 1 shows a partial view of a microsurgical endoprobe 100 including a hand-piece 150 having concentric drive 105, and a cannula assembly 110, according to some embodiments. Endoprobe 100 also includes coupling cable 195, according to some embodiments. Assembly 110 is placed at the distal end of endoprobe 100 and is elongated along the probe's longitudinal axis. Assembly 110 has a limited cross-section, D₂, in order to be minimally invasive in surgery. In some embodiments, cannula assembly 110 is about 0.5 mm in diameter (D₂) while hand-piece 150 may have a substantially cylindrical shape of several mm in diameter (D₁) such as 12-18 mm. The hand-piece dimensions provided above are not limiting. Some embodiments are sufficiently small for an ergonomic fit into the surgeon's hand. For example, some embodiments may have a diameter D₁ of about 10 centimeters (cm), or smaller.

Cable 195 may be included in some embodiments coupling endoprobe 100 to a remote console 198 having a controller device 199. Cable 195 may include power transmission elements to transfer electrical or pneumatic power to concentric drive 105 inside hand-piece 150. For example, electrical power may be provided to concentric drive 105 through drive wires 50 and a ground wiring 20.

Cable 195 may include optical transmission element 40 to carry optical information and power, such as a laser beam, from a remote console or controller to the tissue. Optical transmission element 40 may carry optical information from the tissue to a remote console or controller, for data processing. Element 40 includes one or more optical fibers 40 to transmit light to and from the tissue. In some embodiments, one optical fiber 40-1 may transmit light to the tissue, and another optical fiber 40-2 may transmit light from the tissue. Further, some embodiments may transmit light to and from the tissue through one optical fiber 40. Optical fibers 40 may be single-mode, multimode, or a plurality of single mode and multimode optical fibers. In embodiments of endoprobe 100 used for OCT applications, fiber 40 may be a single mode fiber. In some embodiments, the fiber 40 may be stationary and there may be a gap between the fiber 40 and rotating optical elements (e.g., optical elements 160 or fiber elements 40-1 and 40-2). In some embodiments, the fiber 40 may be attached to and rotate with the first rotating element. A joint on the upstream end of the fiber 40 (e.g., in the handle) may relay light from a stationary fiber into the rotating fiber with a lens.

This may reduce the sensitivity of the inter-relationship between the fiber 40 and the fiber receiving end of endoprobe 100.

Cable 195 may also include switch signal 35. Switch signal 35 may be a signal to turn concentric drive 105 ‘on’ or ‘off,’ according to some embodiments. Signal 35 may include commands to change the operational state of concentric drive 105, such as speed and phase of each of motors 125 and 115. In some embodiments, a button 30 may be included in hand-piece 150 to manually provide signal 35. Button 30 is optional, and may be replaced in some embodiments by a direct control signal carried through cable 195 from remote console 198.

According to some embodiments of endoprobe 100, cable 195 may be absent, and the probe may be wirelessly accessible. In such embodiments, a battery may be included in hand-piece 150 to provide electrical power to motors 115 and 125, and to an optical light source. Further, in embodiments where hand-piece 150 is wireless, hand-piece 150 may include a transceiver device to send and receive data and instructions from the probe to a controller, and vice versa. In such embodiments, hand-piece 150 may also include a processor circuit having a memory circuit to process data, to separately control motors 115 and 125, and control the transceiver device.

Cannula assembly 110 includes inner tube 130 and outer tube 140, arranged concentrically about the probe longitudinal axis (LA). Tubes 130 and 140 are configured to rotate (tube 130) and counter-rotate (tube 140), relative to each other. The definition of rotating and counter-rotating is arbitrary and not limiting of embodiments consistent with the present disclosure. For example, the rotation of tubes 130 and 140 may be defined with respect to a reference. In some embodiments, the reference may be a fixed point in the tissue surrounding assembly 100. At the distal end of cannula assembly 110 optical elements 160 provide light from fiber 40 to the surrounding tissue. Optical elements 160 may also couple light from the tissue into optical fiber 40. Optical elements may include a combination of elements coupled to either one of inner tube 130 and outer tube 140. Thus, in some embodiments, optical elements 160 include components that rotate and counter-rotate relative to each other about the probe LA. The optical axis of elements 160 may be aligned with the probe LA, according to some embodiments. For example, some embodiments may include GRIN (gradient index) lenses as optical elements 160. A GRIN lens may be attached to inner tube 130 and a GRIN lens may be attached to outer tube 140. Further, optical elements 160 may include a prism or a dispersive element attached to a face of each of the GRIN lenses in inner tube 130 and outer tube 140. Dispersive components may be preferred in optical elements 160 for embodiments of cannula assembly 110 used for OCT applications. OCT techniques typically use broadband light sources, thus dispersive elements provide optical performance across the entire bandwidth of the OCT source.

According to some embodiments, a single GRIN lens may be included in optical elements 160. In such embodiments, a GRIN lens may be attached to inner tube 130 having a first prism on the distal face of the GRIN lens. A second prism may be attached to outer tube 140, to provide a deflection mechanism allowing the optical beam to be scanned along a pattern. The opposite configuration is also possible: a first prism attached to inner tube 130 and a GRIN lens coupled to a second prism attached to outer tube 140.

In some embodiments, optical elements 160 may include lenses other than a GRIN lens attached to inner tube 130 and outer tube 140. The two lenses may be configured to form a gap such that the sides of the lenses facing the gap form an angle with respect to the optical axis. Rotating each of the lenses thus configured independently of each other and in opposite directions provides a linear scan of a beam passing through optical elements 160. For example, a linear scan is obtained when the rotating and counter-rotating speeds of the two lenses are equal and opposite to each other relative to a reference. According to embodiments consistent with the present disclosure, a reference may be a fixed point in the tissue surrounding cannula assembly 110. In some embodiments, having a different speed of rotation and of counter-rotation relative to a reference may provide a 2-D scan of a beam passing through optical elements 160. Optical elements 160 may also include a transparent window to prevent contamination of optical elements 160 with materials outside endoprobe 100.

Cannula assembly 110 may include stationary cannula 120. Cannula 120 may provide a protective cover to assembly 110. Also, cannula 120 may prevent or reduce shear strain induced in the target tissue by viscoelastic forces acting upon the rotation of outer tube 140. The use of stationary cannula 120 is optional and may be determined by the type of target tissue where endoprobe 100 will be introduced.

The materials used to form cannula elements 120, 130, and 140 may be any of a variety of biocompatible materials. For example, some embodiments may include elements 120, 130 and 140 made of stainless steel, or plastic materials. Furthermore, some embodiments may have a portion or the entirety of elements 120, 130 and 140 coated with a protective layer. The coating material may be a gold layer, or some biocompatible polymer. In some embodiments, the role of the coating layer may be to provide lubrication and friction relief to moving parts in assembly 110. For example, coating materials may reduce friction between the inner face of tube 140 and the outer face of tube 130. In some embodiments, the role of the coating layer may be to provide protection to the tissue in direct contact with assembly 110.

Embodiments consistent with FIG. 1 may include hand-piece 150 with a removable cannula assembly 110. Assembly 110 may be easily removable from hand-piece 150 by a snap-on mechanism or a bayonet mechanism. Hand-piece 150 may include a bearing and a bushing coupled to the proximal end of tubes 120, 130 and 140 to provide support and stability to assembly 110.

Assembly 110 may be coated with materials that prevent infection or contamination of tissue. Furthermore, surgical procedures and protocols may establish hygienic standards for assembly 110. For example, it may be desirable that assembly 110 be disposed-of after being used once. In some situations, assembly 110 may be disposed-of at least every time the procedure is performed on a different patient or in a different part of the body.

Embodiments of endoprobe 100 and assembly 110 may comply with industry standards such as EN ISO 14971 (2007), “Medical Devices—Application of Risk Management to Medical Devices;” ISO/TS 20993 (2006), “Biological evaluation of medical devices—Guidance on a risk management process;” ISO 14001 (2004), “Environmental management systems—Requirements with guidance for use;” ISO 15752 (2009), “Ophthalmic instruments—endoilluminators—fundamental requirements and test methods for optical radiation safety;” and ISO 15004-2 (2007), “Ophthalmic instruments—fundamental requirements and test methods—Part 2: Light Hazard Protection.”

Table I illustrates a range of dimensions of different elements as labeled in FIG. 1 according to some embodiments. In Table I, ‘ID’ refers to inner diameter, and ‘OD’ refers to outer diameter. Units in Table I are in microns (1 μm=10⁻⁶ m). The dimensions provided in Table I are nominal and can vary in different embodiments depending on the specific application. For example, some embodiments may vary endoprobe dimensions by about 50% from those in Table I. In some embodiments of endoprobe 100 used for ophthalmic microsurgical procedures ‘ODs’ of less than approximately 1 to 1.5 mm may be used.

TABLE I

| Element | OD max | OD min | ID max | ID min |
|---|---|---|---|---|
| 120 | 647.7 | 635 | 609.6 | 571.5 |
| 140 | 546.1 | 533.4 | 495.3 | 469.9 |
| 130 | 419.1 | 406.4 | 381 | 355.6 |
| 40 | 342.9 | 330.2 | 152.4 | 139.7 |

According to embodiments consistent with FIG. 1, the length L₁ of hand-piece 150 is 3-4 inches (approximately 7.5 cm to 10 cm). The length L₂ of cannula assembly 110 is 30 mm. According to some embodiments, cannula assembly 110 may have a portion extending inside hand-piece 150, adding to the length L₂ shown in FIG. 1.

In the embodiment illustrated in FIG. 1, inner tube 130 extends through a central aperture in distal motor 115 and is mechanically coupled to the output of proximal motor 125 by coupler 127. Similarly, outer tube 140 is mechanically coupled to the output of distal motor 115 through coupler 117. Couplers 117 and 127 are positioned concentrically in relation to the probe LA. In some embodiments, an output shaft in proximal motor 125 may pass through a central aperture in distal motor 115 and be coupled to inner tube 130 in a portion of hand-piece 150 distal relative to motor 115.

Concentric drive 105 includes motors 115 and 125 arranged concentrically about the probe LA. In some embodiments, distal motor 115 provides a counter-rotating motion to outer tube 140 and proximal motor 125 provides a rotating motion to inner tube 130. In some embodiments, while tube 130 rotates ‘clockwise,’ tube 140 may rotate ‘counter-clockwise.’ The opposite configuration may occur, wherein tube 130 rotates ‘counter-clockwise’ and tube 140 rotates ‘clockwise.’

Embodiments consistent with the present disclosure, including coaxial motor designs with concentrically nested shafts, reduce the number of transmission elements used. Placing motor elements in line, such as in concentric drive 105, is conducive to the form factor and balance of surgical hand pieces.

In some embodiments, motors 115 and 125 may be electric motors, including continuous electric motors and stepper motors. In some embodiments, motors 115 and 125 may be pneumatic motors (e.g., a piston-cylinder motor). Further, in some embodiments, motors 115 and 125 may be fan or turbine actuated motors (e.g., with a fan turbine propeller). Some embodiments consistent with the present disclosure include at least one of motors 115 and 125 being a piezoelectric motor. A piezoelectric motor used in embodiments consistent with the present disclosure include ratchet-type piezoelectric motors and continuous-type piezoelectric motors driven by high frequency vibrations. Motors 115 and 125 may include an encoder to provide indication of the position of a rotating shaft within the motor relative to a reference. The encoder may be coupled to controller 199 in remote console 198 through cable 195 or wirelessly, according to some embodiments.

Hand-piece 150 is located in the proximal end of endoprobe 100 and has a larger cross section compared to cannula assembly 110. Hand-piece 150 may be adapted for manual operation of endoprobe 100, according to some embodiments. In some embodiments, hand-piece 150 is adapted for robotic operation or for holding by an automated device or a remotely operated device. While cannula assembly 110 may be in contact with living tissue, hand-piece 150 may not be in direct contact with living tissue. Thus, even though hand-piece 150 may comply with hygienic standards, these may be somewhat relaxed as compared to those used for cannula assembly 110. For example, hand-piece 150 may include parts and components of endoprobe 100 that may be used repeatedly before disposal.

Thus, some embodiments of endoprobe 100 as disclosed herein may include complex components in hand-piece 150. Less expensive, replaceable components may be included in cannula assembly 110. Some embodiments may have a removable cannula 110 which is disposable, while hand-piece 150 may be used more than once. Hand-piece 150 may be sealed hermetically, in order to avoid contamination of the tissue with particulates or fumes emanating from internal elements in hand-piece 150. In some embodiments, cannula assembly 110 may be fixed to hand-piece 150 by an adhesive bonding. According to other embodiments, assembly 110 may be removable from hand-piece 150 to allow repeated use of endoprobe 100 in different procedures. Some embodiments consistent with FIG. 1 may have a disposable hand-piece 150 and a disposable assembly 110.

As inner tube 130 and outer tube 140 are counter-rotated relative to each other a light beam passing through elements 160 is deflected from the probe LA. The beam, provided by fiber 40, is deflected at an angle θ given by the specific configuration of optical elements 160. As tubes 130 and 140 rotate and counter-rotate about the probe LA, the light beam completes a full sweep substantially along a line in a plane containing the probe LA. Some embodiments consistent with the above description may use probe 100 in an OCT-scanning procedure. OCT scanning procedures typically include an in-depth image obtained through an A-scan. A collection of A-scans along a line may form a 2-dimensional image in what is referred to as a B-scan. In such cases, as tubes 130 and 140 move they provide a B-scan of the light beam used in OCT imaging.

A B-scan obtained as above may be substantially aligned along a radial direction perpendicular to the probe longitudinal axis (LA) on a projection plane perpendicular to and centered on the probe LA. The specific orientation of the B-scan on the projection plane may be determined by the relative angular phase of rotation and counter-rotation between tube 130 and tube 140. Thus, by adjusting the rotation speed of inner tube 130 and outer tube 140, the radial B-scan formed by the light beam on the projection plane rotates around the probe LA. As a result, in some embodiments the collection of A and B-scans may form a solid section of a cone with its axis along the probe longitudinal axis having an aperture angle θ. This 3-D scan is also known as a C-scan. The angle θ may be the maximum deflection of the light beam for any configuration of elements 160. In some embodiments, this may occur when two faces of prisms or lenses included in elements 160 form opposite angles relative to the probe LA.

In some embodiments, an asynchronous (i.e. out of phase) rotation of inner tube 130 and outer tube 140 provides a 3-D scanning profile, or C-scan. Note that in embodiments consistent with the present disclosure inner tube 130 and outer tube 140 may rotate in the same direction relative to a reference, but at different speeds, to provide a C-scan. A reference may be a fixed point in the tissue surrounding cannula assembly 110. Having independent control of a proximal motor and a distal motor in a concentric drive such as disclosed herein allows endoprobe 100 to perform complex scan patterns. Furthermore, having independent control of a proximal motor and a distal motor in a concentric drive such as disclosed herein allows sweeping a B-scan pattern and forming a 3-D, C-scan pattern. Thus, a rich source of information about the tissue surrounding cannula assembly 110 is provided in embodiments disclosed herein.

Some embodiments using endoprobe 100 for OCT scans may provide a B-scan that is not a perfect line contained within a plane including the probe longitudinal axis. The B-scan provided by endoprobe 100 according to embodiments described above may have a shape resembling an elongated number ‘8’ substantially along a line in a plane containing the probe longitudinal axis. The details of the shape of the B-scan may be determined by parameters such as the optical configuration of elements 160. The shape of the resulting B-scan may also depend on the optical configuration of elements 160. Also, the shape of the B-scan may be determined by the indexes of refraction of optical elements 160 and of the material embedding elements 160.

In embodiments consistent with the present disclosure used for OCT applications, endoprobe 100 may be configured for an operator to select between an A-scan, a B-scan, or a C-scan. For example, an A-scan may be obtained by having inner tube 130 and outer tube 140 stopped, disengaging concentric drive 105. A B-scan may be provided by setting motor 115 and 125 moving at equal speeds in opposite directions. Moving motors 115 and 125 at different speeds may result in a C-scan. These operations may be controlled from remote console 198 or with push button 30.

Accordingly, embodiments consistent with the present disclosure include a drive mechanism such as concentric drive 105 providing independent rotation control of cannula tubes 130 and 140. Cannula tubes 130 and 140 include scanning elements such as optical elements 160. The cannula tubes 130 and 140 are nested concentrically about the probe LA where the outer tube 140 is attached or integrated into the (hollow) shaft of a distally placed electric motor 115. The inner tube 130 is attached to or integrated into the (hollow) shaft of a proximally placed electric motor. The hollow shaft passes through an aperture in distal motor 115. In some embodiments, the inner tube 130 may go all the way through distal motor 115 and be coupled to proximal motor 125 through coupler 127.

In some embodiments, stepper motors are used as motors 115 and 125, and the angular positions of the motor shafts are controlled independently from one another using different encoders for proximal motor 125 and distal motor 115.

Some embodiments consistent with the present disclosure may use a slightly eccentric configuration of elements 160 in order to provide a B-scan trajectory forming a loop similar to an elongated ellipse. For example, elements 160 may include a proximal optical element attached to inner tube 130 and a distal optical element attached to outer tube 140, such that the proximal optical element has an optical axis slightly off the mechanical axis of inner tube 130. In such configuration, a trajectory of an optical beam makes a loop travelling along an upper portion in one half of the trajectory and completing a lower portion of the second half of the trajectory. Such configuration essentially doubles the speed of a volume acquisition during a C-scan. Also, some embodiments may use such configuration of elements 160 to provide a spatial dual scan differentiation. For example, surface gradient measurements may be obtained using a slightly eccentric configuration for elements 160. The eccentricity of proximal and distal optical elements may be chosen appropriately for different applications. For example, some applications having proximal and distal optical elements with diameters between about 0.5 mm to about 1 mm use a proximal optical element having its optical axis shifted by about 100 μm (=0.1 mm) from the mechanical axis of assembly 110, such as the long axis (LA) of endoprobe 100. Embodiments consistent with the present disclosure may introduce an eccentricity into the configuration of elements 160 by mechanically placing a proximal element off-center from the LA of endoprobe 100, attached to inner tube 130. In some configurations, it is a distal element in the configuration of elements 160 that is attached to outer tube 140 off-center from the LA of endoprobe 100. Some embodiments may place proximal and a distal elements 160 concentric about the LA of endoprobe 100, but with either one of the proximal or distal elements having an optical axis off-center from the LA of endoprobe 100. For example, a rod style GRIN lens may be built with the optical axis offset from the LA of endoprobe 100 and used as a proximal or a distal element in configuration 160. In some embodiments, a conventional or aspheric lens may be used as one of elements 160, and an eccentricity may be provided by grinding the lens so that the optical axis is off-center relative to the LA of endoprobe 100. Further embodiments may include a conventional, aspheric, or a GRIN lens cut in a cylindrical shape having a geometrical center offset from the center of the cylinder.

Embodiments as disclosed herein enable 2-D lateral scanning of an optical excitation beam without having to increase the number of probe tubes. Thus, virtually the entire cross section of cannula assembly 110 (D₂) may be used for collection of the optical signal from the tissue. This provides a better signal-to-noise ratio for a given endoprobe size. In turn, the overall cross-section of the distal end of endoprobe 100 may be reduced as compared to prior art probes thus limiting the incision size on a patient. Embodiments consistent with the present disclosure enable the design of a small gauge endoprobe 100, which is desirable for ophthalmic surgical applications. Incision wounds may then be easier to manage and control for after-surgery recovery.

Embodiments of endoprobe 100 consistent with the present disclosure reduce the number of gears and transmission elements that may be included inside hand-piece 150 to accomplish counter-rotating tubes in a cannula assembly. Each motor 115 and 125 provides independent motion to tubes 140 and 130, respectively, simplifying the mechanical design and the total friction in the system.

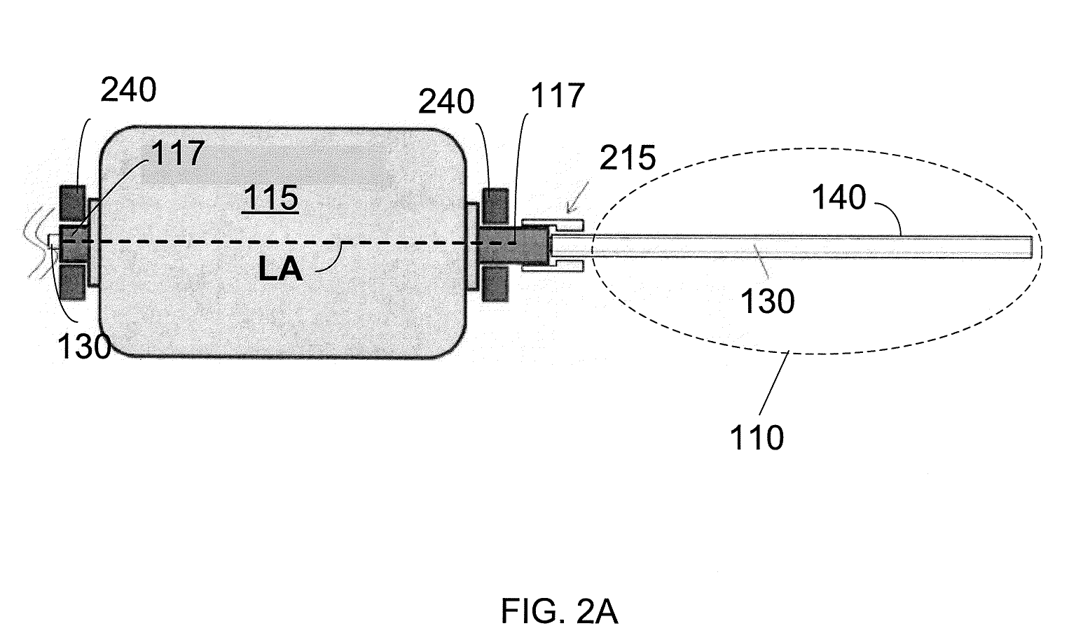

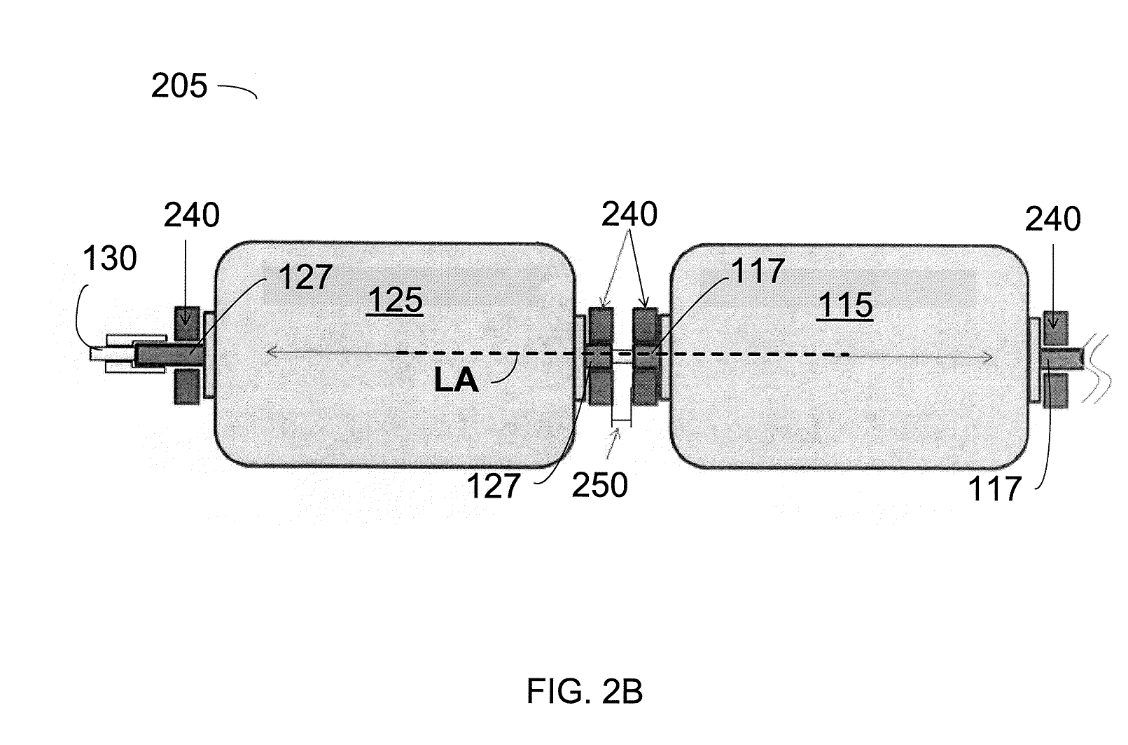

FIG. 2A shows a partial view of a distal motor 115 and a cannula assembly 110 according to some embodiments. In FIG. 2A distal motor 115 is attached to outer cannula 140 through coupler 117. Coupler 117 includes fixture 215. In addition, the motor shaft is hollow, allowing inner tube 130 to pass through outer tube 140 and the motor shaft. Inner tube 130 may thus spin freely within cannula assembly 110.

In some embodiments, tubes 130 and 140 may be integrated into the shafts of motors 125 and 115 as one piece, respectively. This reduces the number of parts and difficulty of assembly. For example, this can be done by directly attaching compact permanent magnets to tubes 130 and 140 so that they are part of the rotors in motors 125 and 115, respectively.

FIG. 2B shows a partial view of a concentric drive 205 according to some embodiments. Proximal motor 125 is attached to inner tube 130, through coupler 127. In some embodiments, inner tube 130 may be integrated into the shaft of motor 125. Alternatively, the inner diameter of the motor shaft in motor 125 may be smaller than the diameter of inner tube 130 so that inner tube 130 is press-fit into the motor shaft. In embodiments consistent with the present disclosure, fiber 40 is placed through inner tube 130 and held fixed in place. Concentric drive 205 includes bushings 240 to maintain the axes of motors 125 and 115 in place, and couplers 117 and 127, as described above in relation to FIG. 1. A gap 250 between motor 115 and 125 ensures that the motor shafts may rotate freely from one another, avoiding contact between motors 125 and 115.

According to embodiments consistent with the present disclosure where motors 115 and 125 are electric motors, several configurations may be used to provide separate rotation of tubes 130 and 140. While tubes 130 and 140 are rotated separately, their motion may be commonly controlled from console 198 through controller 199. As shown in FIG. 2B motors 115 and 125 may be two identical electric motors oriented co-axially along the probe LA, in opposite directions. Thus, by powering motor 115 outer tube 140 rotates in one direction, and while powering motor 125 inner tube 130 rotates in the opposite direction. In some embodiments, motors 115 and 125 may be identical and oriented coaxially with the probe LA, in the same direction. In this configuration, by applying a voltage V to motor 115 outer tube 140 rotates in one direction, and by applying a voltage −V to motor 125 inner tube 130 rotates in the opposite direction. Further embodiments may be configured to rotate tubes 130 and 140 in opposite direction according to the winding of an internal coil in each of electric motors 115 and 125.

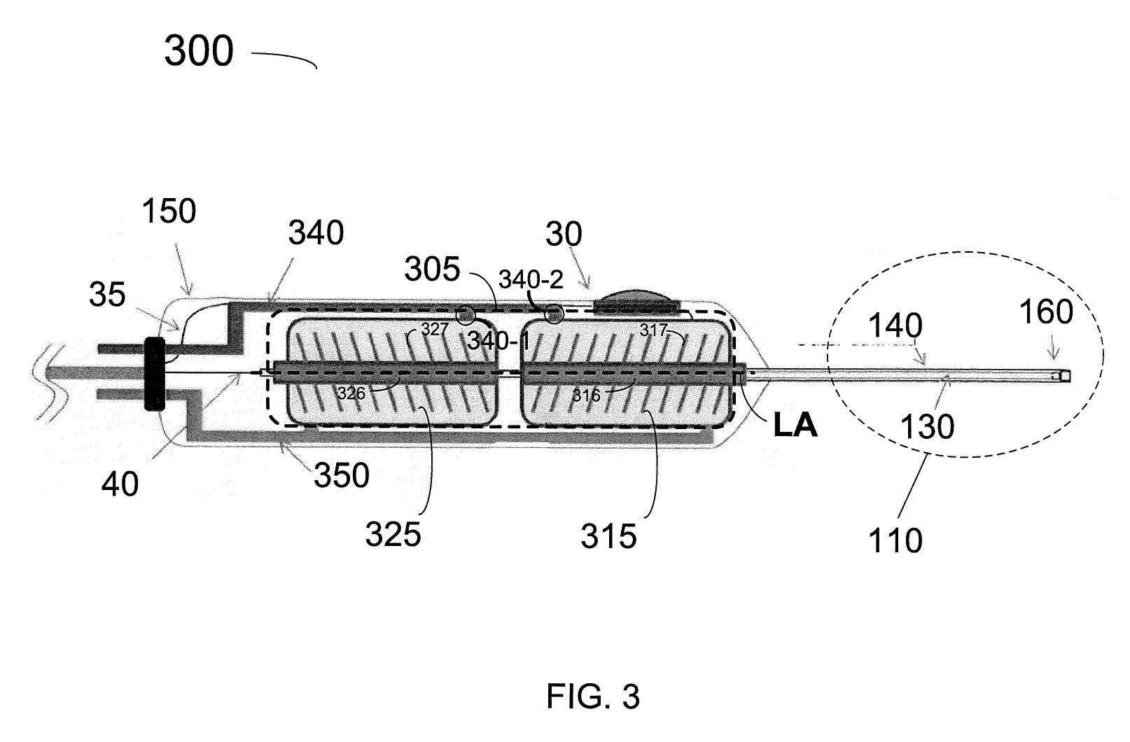

FIG. 3 shows a partial view of a microsurgical endoprobe 300 including a hand-piece 150 having concentric drive 305 and cannula assembly 110, according to some embodiments. Concentric drive 305 includes motors 325 and 315 using fan turbine propellers. In some embodiments, a single fluid channel 340 provides power to the two motors. The fluid may be air, another gas, or may be a liquid. In some embodiments, channel 340 may include separate fluid channels 340-1 and 340-2 for independent drive control of each motor 315 and 325, respectively. According to endoprobe 300, outer tube 140 is attached to the main drive shaft 316 of distal motor 315 through a mechanical coupler such as coupler 215 (see e.g., FIG. 2A). In some embodiments, tube 140 may be integrated into drive shaft 316 as one piece or two or multiple press-fit pieces. Inner cannula 130 is attached to the main drive shaft 326 of proximal motor 325. Accordingly, a single endoprobe as described herein may include scanning tubes 130 and 140 attached to, or integrated into the drive shaft of fan or micro-turbine motors 325 and 315, respectively.

In embodiments consistent with the present disclosure, drive shafts 316 and 326 are coupled to turbines 317 and 327, respectively. Turbines 317 and 327 may be helicoid propellers, threaded such that as a fluid passes through motors 315 and 325, shafts 326 and 316 rotate about the probe LA. The fluid is provided by input channel 340, which is pressurized to allow flow through motors 315 and 325. The fluid exits concentric drive 305 through exhaust channel 350. According to some embodiments, turbines 317 and 327 are threaded such that drive shafts 316 and 326 rotate in opposite directions relative to a reference. The reference may be a fixed point in the tissue surrounding cannula assembly 110. In some embodiments, input channel 340 may include two separate channels 340-1 and 340-2 to feed each of motors 315 and 325 independently of one another. In such configurations, the threading of turbines 327 and 317 may be the same, while still providing a rotating and counter-rotating motion to drive shafts 326 and 316, respectively. For example, input channels 340-1 and 340-2 may be configured such that the fluid flows in opposite directions in each motor 325 and 315. According to methods consistent with the present disclosure, exhaust channel 350 is common to motors 315 and 325, thus reducing size and complexity of endoprobe 300.

The control of the rotation speed in motors 315 and 325 may be provided by valves (not shown) placed in input channel 340. The valves may independently control the flow from input channel 340 to each of motors 315 and 325, thus adjusting the rotational speed of tubes 130 and 140 independently. In some embodiments, independent flow channels 340-1 and 340-2 may provide different flow speeds according to controls provided in a remote console. The flow on channels 340-1 and 340-2 may be adjusted with valves located in console 198 via controller 199.

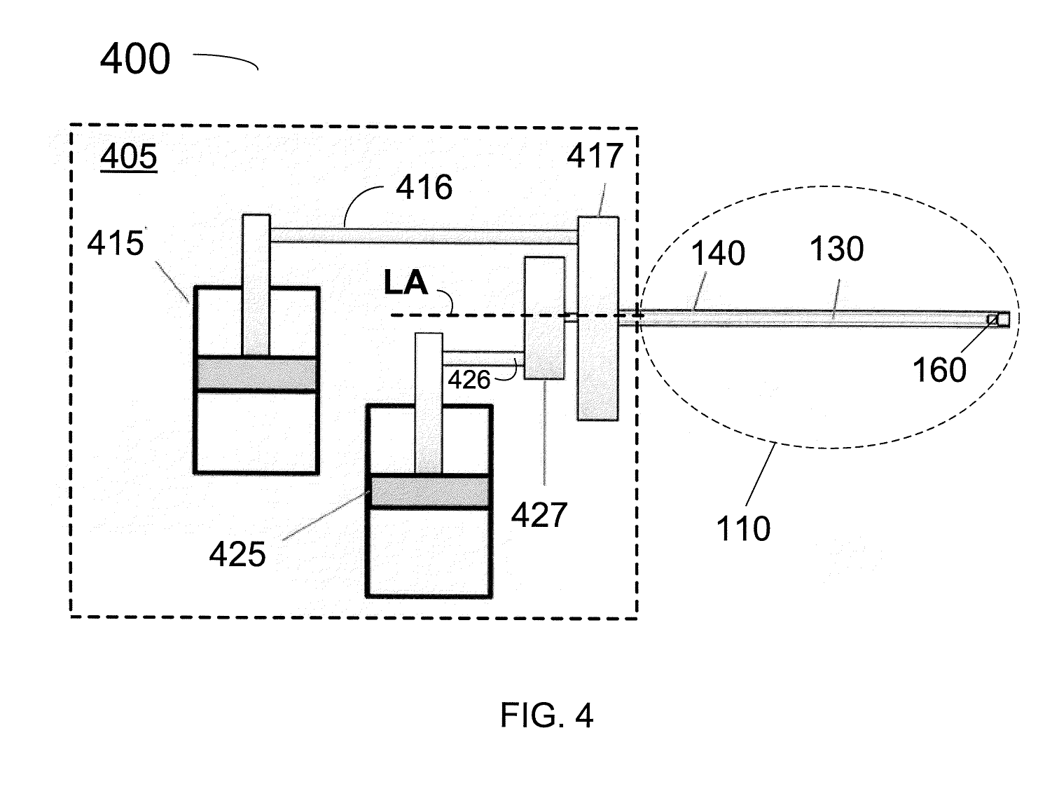

FIG. 4 shows a partial view of an endoprobe 400 including concentric drive 405 and cannula assembly 110, according to some embodiments. Concentric drive 405 includes reciprocating pneumatic piston-cylinder motors 415 and 425 to drive cannula assembly 110. FIG. 4 shows each motor 415 and 425 powering cannula tubes 140 and 130 through concentrically aligned crankshafts 417 and 427, respectively. Outer tube 140 is attached to proximal motor 415 through shaft 416 and crankshaft 417. Inner tube 130 is attached to distal motor 425 through shaft 426 and crankshaft 427. Accordingly, endoprobe 400 as disclosed herein includes scanning tubes 130 and 140 attached to, or integrated into crankshafts 427 and 417 of reciprocating pneumatic motors 425 and 415, respectively.

Embodiments such as endoprobe 400 may be used to provide a reciprocating drive to cannula assembly 110. In such configuration, inner tube 130 and outer tube 140 may perform a fractional rotation before reversing directions. Furthermore, some embodiments of endoprobe 400 may be such that inner tube 130 performs a full rotation while outer tube 140 performs a fractional rotation. Embodiments such as endoprobe 400 have the advantage that outer tube 140 may be placed in direct contact with the surrounding tissue or vitreous humor. By performing a fractional rotation in a reciprocating motion (back-and-forth), outer tube 140 may reduce the friction and tear stress on the tissue or vitreous humor. This in turn may eliminate the need for outer tube 120 in cannula assembly 110, thus reducing the overall cross-section of the probe.

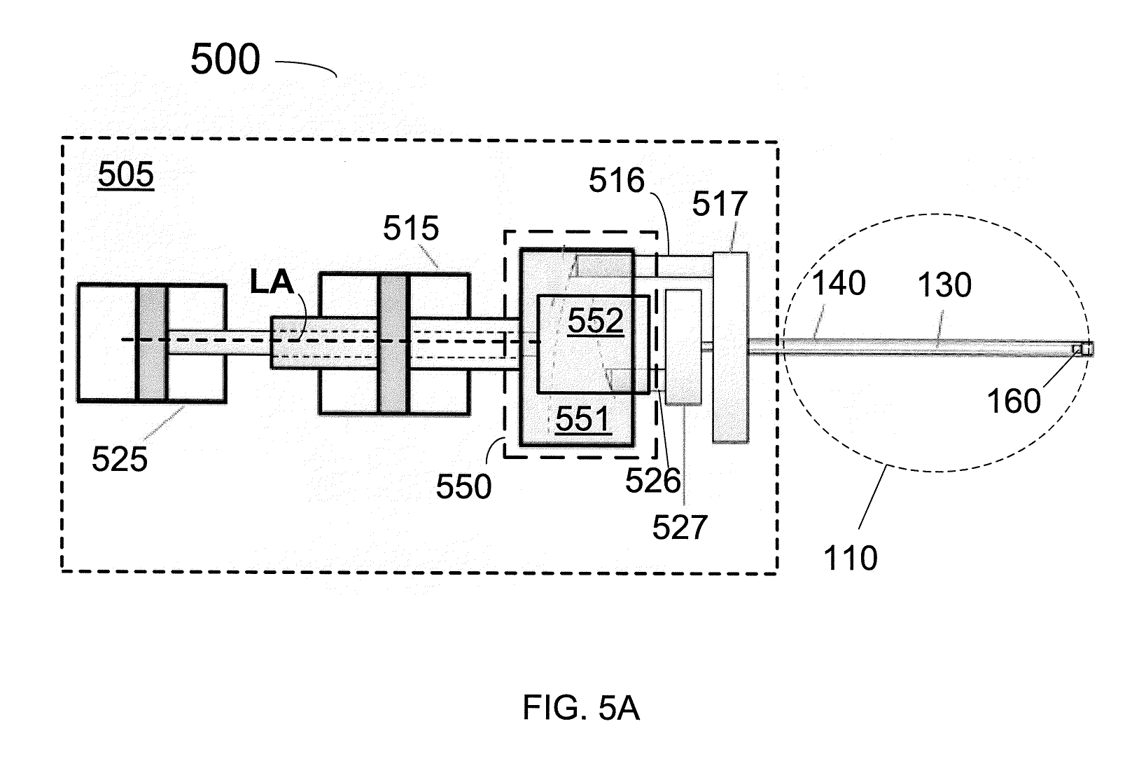

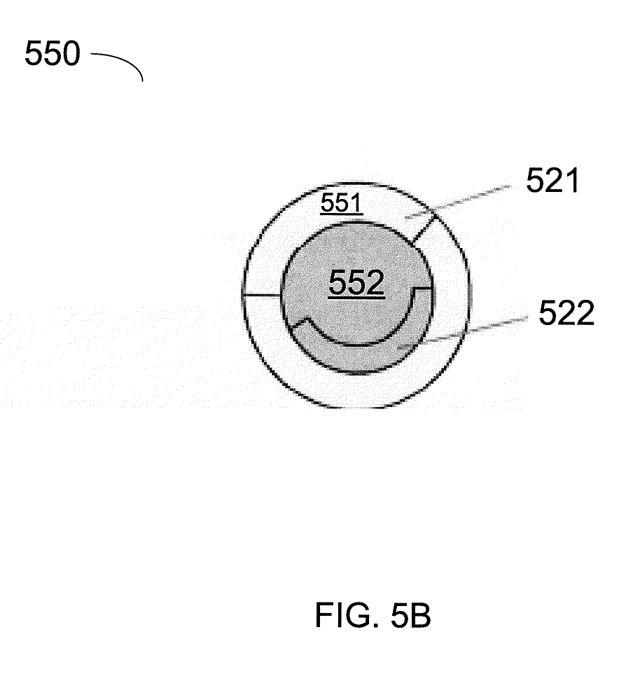

FIG. 5A shows a partial view of an endoprobe 500 including concentric drive 505 and cannula assembly 110, according to some embodiments. Endoprobe 500 includes concentric drive 505 with concentrically mounted piston cylinder motors 525 and 515. Geometry such as in concentric drive 505 provides a compact endoprobe 500, which is desirable for delicate surgical procedures. Concentric drive 505 includes slider 550 to couple the horizontal motion of the piston shafts in 525 and 515 to the rotational and counter rotational motion of crankshafts 517 and 527. Cannula tubes 130 are mounted or inserted on crankshafts 527 and 517, respectively.

Slider mechanism 550 is a nested double cylinder to provide independent control of crankshaft 517 and 527, respectively. Slider 550 includes cylinder 551 coupled to motor 515 and cylinder 552 coupled to motor 525. Cylinders 551 and 552 move independently of each other. As cylinder 551 moves back-and-forth (left-right direction in FIG. 5A), it pushes shaft 517. As cylinder 552 moves back-and-forth it pushes shaft 526. As shafts 516 and 517 are pushed they ‘slide’ off in a perpendicular direction to the slider motion. That is, according to FIG. 5A, as cylinder 551 moves to the right, shaft 516 slides off in a direction ‘out’ of the plane of the figure. Likewise, as cylinder 552 moves to the right, shaft 526 slides off in a direction ‘into’ the plane of the figure. Some embodiments may include return springs coupling each of shafts 516 and 526 to slider 550 to return crankshafts 517 and 527 to their original positions. In some embodiments, shafts 516 and 526 may have a ‘tongue’ that fits into a groove etched on the interior faces of cylinders 551 and 552. In order to provide a rotational motion in opposite directions for each of crankshafts 517 and 527, slider 550 may have two ‘ledgers’ or grooves etched clock and counter-clock wise on its interior face.

Note that the slider mechanisms are integral parts of concentric drive 505; therefore, the use of a transmission mechanism is avoided in some embodiments. Slider 550, shafts 516 and 526, and crankshafts 517 and 527 may be molded, reducing manufacturing costs. In FIG. 5A the size of slider 550 is exaggerated for diagrammatic purposes. Some embodiments may include a miniaturized version having diameters of a few millimeters. In some embodiments, slider 550 includes a single piece having two ‘ledgers’ or grooves etched in clock and counter-clock directions, on an inner face. Thus, a 1-D scanning using a single piston motor may be implemented according to some embodiments. The slider would then have two angled surfaces which would drive both crankshafts simultaneously.

FIG. 5B shows a partial cross sectional view of slider coupling mechanism 550 according to some embodiments. Slider 550 includes cylinders 551 and 552. Cylinder 551 may have a groove or ‘ledger’ 521 formed in its interior face. Cylinder 552 may have a groove or ‘ledger’ 522 formed in its interior face. In some embodiments consistent with the present disclosure, ledger 521 may thread clockwise and ledger 522 may thread counter-clockwise relative to slider 550. For example, the left side of ledger 521 may be closer to the viewer than the right side (deeper), in FIG. 5B. Likewise, the left side of ledger 522 may be farther from the viewer (deeper) than the right side, in FIG. 5B.

In embodiments consistent with the present disclosure, cylinders 551 and 552 may slide relative to each other, so that their motion is independent from one another. Cylinder 551 may be moved (in and out of the plane in FIG. 5B) at a certain speed by motor 515 (see e.g., FIG. 5A). Cylinder 552 may be moved (in and out of the plane in FIG. 5B) at a different speed and with a different phase by motor 525 (see e.g., FIG. 5A). Furthermore, the phase of cylinders 551 and 552 may be adjusted by separately controlling motors 515 and 525.

In some embodiments, cylinders 551 and 552 may be fixed to each other, and powered by a single motor. In this case, the rotating and counter-rotating motion of crankshafts 527 and 517 will be synchronous and have a fixed phase. Such a configuration may provide a 1-D scan by optical elements 160 attached to cannula assembly 110.

Furthermore, in order to minimize abrasion to the tissue or vitreous humor in direct contact with cannula assembly 110, some embodiments of a concentric drive as disclosed herein provide a ‘spooling’ motion. A ‘spooling’ motion is such that tubes 130 and 140 rotate in one direction for one cycle, and switch to rotate in the opposite direction in the next cycle. Thus, while the scanning effect is still a linear trajectory, the tissue surrounding assembly 110 is subjected to reduced shear. Furthermore, in a spooling configuration, cannula tubes 130 and 140 may not complete a 360° rotation in each cycle, thus minimizing shear of surrounding tissue or vitreous humor.

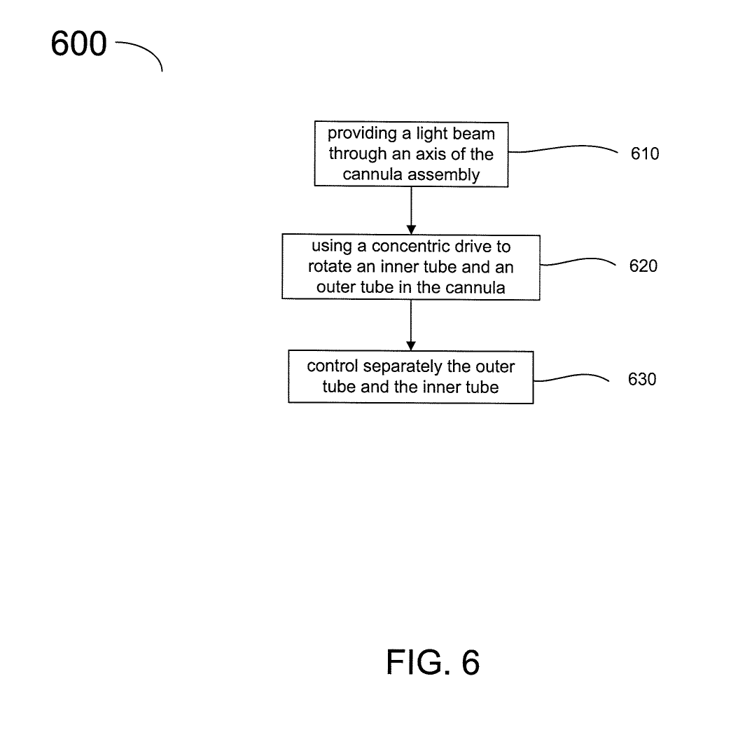

FIG. 6 shows a flow chart of a method 600 for scanning a light beam using a cannula assembly according to some embodiments. Method 600 may be performed by a user controlling a microsurgical endoprobe as disclosed herein. According to some embodiments, the microsurgical endoprobe may be operated manually by the user, or robotically. Further, part of method 600 may be partially performed by the user and part may be partially performed through controller 199 in console 198. Method 600 includes 610 for providing a light beam through an axis of the cannula assembly. At 620, a concentric drive in a hand-piece proximal to the cannula may be used to provide a rotation to an inner tube and a rotation to an outer tube in the cannula. According to embodiments consistent with the present disclosure each of the outer tube and inner tube is hollow and has an optical element in its distal end. At 630, the rotation of the outer tube and the rotation of the inner tube are controlled separately from each other, using the concentric drive.

In some embodiments consistent with the present disclosure, providing a light beam in 610 may include using an optical fiber or a plurality of optical fibers 40 (see e.g., FIG. 1) to carry a laser light through the fiber, or a laser pulse, or a light provided by a lamp coupled to fiber 40. At 620, in some embodiments a microsurgical probe such as 100, 300, or 500 (see e.g., FIGS. 1, 3 and 5A above) may be used. Thus, a concentric drive in 620 may include a proximal and a distal electric motor (see e.g., FIG. 1), or a proximal and a distal pneumatic motor (see e.g., FIG. 3), or a proximal and a distal turbine fan motor (see e.g., FIG. 5A).

According to embodiments consistent with the present disclosure, a concentric drive may provide a rotation and counter-rotation speed to tubes 130 and 140 varying form 1 Hertz (Hz) (one turn per second) up to 1 kilohertz (kHz) (one thousand turns per second) or more.

In some embodiments, the rotating and counter rotating speeds of tubes 130 and 140 may be substantially higher, such as 8200 rotations per minute (RPM) or more. For example, according to embodiments herein, a fast rotation speed may be desired for endoprobes used for OCT scanning. In such cases, the maximum speed of rotation of tubes 130 and 140 may be limited by the detector acquisition speed in the OCT scanner. Furthermore, some embodiments using a ‘spooling’ motion may use rotating and counter-rotating speeds for tubes 130 and 140 at a higher speed compared to configurations using continuous motion. This is due to the higher tolerance of the surrounding tissue or vitreous humor for shear and stress in a ‘spooling’ configuration. Endoprobes according to embodiments disclosed herein used in OCT scanning may include a ‘spooling’ motion rotating at twice the speed of a configuration using a continuous motion to complete the same B-scan. A high rotational speed may be desirable in OCT-scanning embodiments in order to produce 3D volume imaging. OCT-scanning systems may provide A-scans at rates varying from about 25 kHz up to 400 kHz, thus the rotation speeds of tubes 130 and 140 provided by concentric drives 105, 305, 405 and 505 may be substantially lower than those values. Furthermore, in some embodiments consistent with the present disclosure tubes 130 and 140 may rotate in a step-and-scan configuration. In such cases, concentric drives 105, 305, 405, and 505 may move a probe beam from one location to another, and wait for an A-scan to be completed before moving the probe beam to a different location. In some embodiments, a plurality of A-scans may be collected at each point before moving the probe beam to a different location, to perform averaging and error correction at each point.

A probe according to embodiments disclosed herein may provide a simple, efficient mechanism to generate precisely controlled counter rotational motion in two concentric tubes. Such a probe may be used as an OCT imaging probe, or a multi-spot laser probe. While probes may have 3-dimensional layouts, they may be highly constrained in cross-section and elongated in a certain direction. Furthermore, in some embodiments the probes may be axially symmetric, at least in a portion of the probe which may include the distal end.

In OCT imaging techniques, a light beam having a coherence length may be directed to a certain spot in the target tissue by using a probe. The coherence length provides a resolution depth, which when varied at the distal end of the probe may be de-convolved to produce an in-depth image of the illuminated portion of the tissue (A-scan). A 2-D tissue image may be obtained through a B-scan. In some embodiments, B-scans are straight lines along a cross-section of the tissue. Furthermore, by performing repeated B-scans along different lines in the tissue, a 3-D rendition of the tissue may be provided (C-scan). In some embodiments, the B-scans may be a set of lines having the same length and arranged in a radius from a common crossing point. Thus, the plurality of B-scans provides an image of a circular area in the tissue, having a depth.

According to some embodiments of endoprobe 100 used for OCT-imaging, a plurality of A-scans may be completed for each B-scan step. For example, 512 A-scans may be used to complete one B-scan. Some embodiments may use a lower number of A-scan per B-scan cycle, thus allowing the B-scan procedure to take place at a faster rate. In such cases, the rotating and counter-rotating speeds of tubes 130 and 140 may be further increased.

An endoprobe having a concentric drive as disclosed herein may provide a complex set of scan lines, including B-scan lines arranged in pre-selected patterns. Tubes 130 and 140 may include delicate optical components 160 moved to steer a light beam along a desired direction. Precise control of this motion aids the efficacy of OCT procedures. In particular, repeatability of the motion may be useful for A-scans to be aligned along B-scan lines to conform a continuous image. In some embodiments, the motion of movable parts in the probe may be a periodic cycle having a closed trajectory. For example, a trajectory may be circular, centered on the probe LA. The probe LA may be the optical axis of an optical system.

A substantially one dimensional probe having a symmetry axis according to some embodiments disclosed herein may provide a radial-oriented B-scan about the probe LA. To achieve this, counter-rotating tubes 130 and 140 may be used with concentric drive 105 rotating motor 125 and counter-rotating motor 115 synchronously. For example, counter-rotating tubes 130 and 140 may provide optical scanning of a beam along a radial direction in a plane perpendicular to and centered on the probe longitudinal axis. Such an arrangement may use optical elements as described in detail in the paper by Wu et al. (J. Wu, M. Conry, C. Gu, F. Wang, Z. Yaqoob, and C. Yang; “‘Paired-angle-rotation scanning optical coherence tomography forward-imaging probe” Optics Letters, 31(9) 1265 (2006)). A concentric drive according to embodiments disclosed herein may be configured to adjust the relative phase and speed of tubes 130 and 140 as desired. Thus, tubes 130 and 140 may provide linear radial scanning along a plane including the probe LA.

By adjusting the relative angular speeds and phases of proximal and distal motors included in a concentric drive as disclosed herein the plane of the radial scan may be rotated about the probe LA. Some embodiments consistent with the present disclosure may be such that the radial scan is not perfectly linear. That is, the optical beam may not move in a straight line contained within a plane including the probe LA. In some embodiments, the trajectory may form an elongated loop substantially close to a line in a plane including the probe LA. In some embodiments, the trajectory of the optical beam may form an elongated ‘8’ figure on a plane perpendicular to and centered on the probe longitudinal axis.

In some embodiments, OCT techniques use forward-directed scan procedures. In this case, optical illumination takes place in the forward direction of the probe longitudinal axis. In forward-directed scans, the target tissue may be ahead of the probe in a plane perpendicular to the probe LA. Thus, light traveling from the tip of the probe to the tissue, and back from the tissue into the probe may travel in a direction substantially parallel to the probe longitudinal axis. In embodiments using forward-directed scans the target tissue may be approximately perpendicular to the probe longitudinal axis, but not exactly. Furthermore, in some embodiments light traveling to and from the target tissue from and into the probe may not be parallel to the probe longitudinal axis, but form a symmetric pattern about the probe longitudinal axis. For example, light illuminating the target tissue in a forward-directed scan may form a solid cone or a portion thereof about the probe longitudinal axis. Likewise, light collected by endoprobes in a forward-directed scan consistent with the present disclosure may come from target tissue in a 3-D region. A 3-D region according to some embodiments may include a conical section around the probe LA.

In some embodiments, an endoprobe as provided herein may be used to deliver laser light for therapeutic purposes. For example, in photodynamic procedures a laser light may be scanned to activate a chemical agent present in a drug previously delivered to the target tissue. In some embodiments, laser light may be used to selectively oblate or remove tissue or residual materials from the target areas. In embodiments such as previously described, precise control of the light being delivered is provided by movable components in the distal end of the probe. Thus, use of a concentric drive as disclosed herein allows for independent control of each of the movable components.

Embodiments of the invention described above are exemplary only. One skilled in the art may recognize various alternative embodiments from those specifically disclosed. Those alternative embodiments are also intended to be within the scope of this disclosure.

What is claimed is:

1. An endoprobe for microsurgical procedures, comprising:
a hand-piece having a concentric drive comprising a proximal coupling and a distal coupling;
a cannula assembly coupled to the hand-piece;
the cannula assembly comprising:
an outer tube having a longitudinal axis;
an inner tube positioned within the outer tube, the distal coupling providing a first rotation to the outer tube about the longitudinal axis and the proximal coupling providing a second rotation to the inner tube within the outer tube.

2. The endoprobe of claim 1, wherein the hand-piece includes a first motor coupled to the distal coupling to provide the first rotation and a second motor coupled to the proximal coupling to provide the second rotation.

3. The endoprobe of claim 2, further including a controller coupled to the first motor and the second motor, wherein the first motor provides a rotation to the inner tube in one direction and the second motor provides a rotation to the outer tube in an opposite direction.

4. The endoprobe of claim 1, wherein the proximal coupling and the distal coupling are controlled independently of each other.

5. The endoprobe of claim 1, wherein the proximal coupling and the distal coupling are concentrically located about a probe longitudinal axis.

6. The endoprobe of claim 1, wherein the cannula assembly comprises a stationary tube concentric and exterior to the outer tube.

7. The endoprobe of claim 1, wherein the microsurgical procedures involve the use of light, the endoprobe comprising:
a first optical element attached to the outer tube and a second optical element attached to the inner tube; and
wherein the rotation of the outer tube and the inner tube provides a scanning of a light beam.

8. The endoprobe of claim 7, wherein at least one of the optical elements is placed with its optical axis off-center from the longitudinal axis.

9. The endoprobe of claim 7, wherein the inner tube rotates at a first speed relative to a reference and the outer tube rotates at a second speed relative to the reference.

10. The endoprobe of claim 7, wherein the first and second optical elements comprise two lenses.

11. The endoprobe of claim 10, wherein the two lenses define a gap, the sides of the lenses facing the gap forming an angle relative to an optical axis.

12. The endoprobe of claim 11, wherein at least one of the two lenses is a GRIN (gradient index) lens.

13. The endoprobe of claim 7, wherein the optical elements comprise at least one prism or at least one dispersive element.

14. The endoprobe of claim 1, wherein the concentric drive comprises reciprocating pneumatic piston-cylinder motors to drive the cannula assembly.

15. The endoprobe of claim 1, wherein the microsurgical procedures comprise optical coherence tomography, single spot and multi-spot therapeutic laser delivery, and illumination of tissue.

16. A method for scanning a light beam using a cannula assembly, comprising:
providing a light beam through an axis of the cannula assembly;
using a concentric drive in a hand-piece proximal to the cannula to provide a rotation to an inner tube and a rotation to an outer tube in the cannula; wherein each of the outer tube and inner tube is hollow and has an optical element in its distal end; and
controlling separately the rotation of the outer tube and the rotation of the inner tube using the concentric drive.

17. The method of claim 16, wherein controlling separately a rotating speed includes rotating the inner tube at a first speed relative to a reference and rotating the outer tube at a second speed relative to the reference.

18. The method of claim 17, including scanning an optical beam along a 1-D path in a tissue surrounding the cannula assembly.

19. The method of claim 16, including scanning an optical beam along a 2-D path in a tissue surrounding the cannula assembly.

20. The method of claim 16, including:
providing light to a 3-D portion of a tissue surrounding the cannula assembly; and
collecting light from the 3-D portion of the tissue.
